(19) United States
(12) Patent Application Publication
(10) Pub. No.: US 2010/0274264 A1
(43) Pub. Date: Oct. 28, 2010

(54) Surgical instrument for the placement of ligature clips

(75) Inventors: Peter Schulz, Loeffingen (DE); Pedro Morales, Tuttlingen (DE); Dieter Weisshaupt, Immendingen (DE)

Correspondence Address: Lipsitz & McAllister, LLC, 755 MAIN STREET, MONROE, CT, 06468, US

(73) Assignee: Aesculap AG, Tuttlingen (DE)

(21) Appl. No.: 12/798,317

(22) Filed: Mar. 31, 2010

(30) Foreign Application Priority Data
Apr. 24, 2009 (DE) 10 2009 018 819.3

(51) Int. Cl. A61B 17/10

(52) U.S. Cl. 606/143

(57) ABSTRACT

In a surgical instrument for placement of C-shaped ligature clips having two legs running next to one another and connected to one another by means of a bridge-section, with two clamping jaws movable relative to one another, with a supply guide means, along which the ligature clip can be pushed onto the clamping jaws with the free ends of the legs first, with a clamping jaw guide means, which adjoins the supply guide means and in which the ligature clip can be inserted between the clamping jaws, and with a feeding means for advancing a ligature clip out of the supply guide means into the clamping jaw guide means and into an end position between the clamping jaws, in order to improve the guidance of the ligature clips during transfer from the supply guide means into the clamping jaw guide means, it is proposed that the feeding means comprises a feed element, which can be placed against the ligature clip to be fed and is guided in a longitudinal guide means running along its feed path in the transition region from the supply guide means into the clamping jaw guide means.

This application claims the benefit of German Patent Application No. 10 2009 018 819.3 filed on Apr. 24, 2009.

The present disclosure relates to the subject matter disclosed in German patent application No. 10 2009 018 819.3 of Apr. 24, 2009, which is incorporated herein by reference in its entirety and for all purposes.

### BACKGROUND OF THE INVENTION

The invention relates to a surgical instrument for the placement of C-shaped ligature clips having two legs running next to one another and connected to one another by means of a bridge section, with two clamping jaws movable relative to one another, with a supply guide means, along which the ligature clip can be pushed onto the clamping jaws with the free ends of the legs first, with a clamping jaw guide means, which adjoins the supply guide means and in which the ligature clip can be inserted between the clamping jaws, and with a feeding means for displacing a ligature clip out of the supply guide means into the clamping jaw guide means and into an end position between the clamping jaws.

Such a surgical instrument is described, for example, in patent document EP 0697198 B1. In this case, the clamping jaw guide means directly adjoins the supply guide means and is relatively short. Moreover, the clamping jaw guide means in known instruments is either aligned with the supply guide means or is inclined only slightly in relation to this. Therefore, it is possible to advance the ligature clip relatively easily from the supply guide means into the clamping jaw guide means solely by means of a feed element acting on the rear distal end of the ligature clip.

However, a disadvantage with these instruments is that the placement region of the ligature clips between the clamping jaws is relatively poorly visible for the surgeon, since it directly adjoins the supply guide means, which is usually located in a cartridge accommodating a number of ligature clips in a shank of the instrument and also projects only slightly laterally beyond this shank because of the very slight inclination. It would be desirable to incline the clamping jaw guide means relatively steeply in relation to the supply guide means and also arrange this at a greater distance in front of the supply guide means, so that the surgeon can readily see into this region.

The object forming the basis of the invention is to configure a surgical instrument of the above-mentioned type so that an optimum guidance and an optimum feed of the ligature clips is also assured in this case.

### SUMMARY OF THE INVENTION

This object is achieved according to the invention with a surgical instrument of the above-described type in that the feeding means comprises a feed element, which can be placed against the ligature clip to be fed and is guided in a longitudinal guide means running along its feed path in the transition region from the supply guide means into the clamping jaw guide means.

While in known devices the feed element runs freely at its front end that can be placed against the ligature clip, according to the present invention it is provided that in this front region of the feed element it is guided in its own guide means, which follows the geometry of the supply guide means and the adjoining clamping jaw guide means, so that over the entire feed path the now guided feed element abuts securely against the ligature clip, even in the case of a long feed path and a steeply inclined feed path.

In particular, it can be provided that the longitudinal guide means is formed by at least one longitudinal groove, into which projects a lateral guide projection of the feed element.

It is particularly advantageous if a respective longitudinal groove, into which lateral guide projections of the feed element located opposite one another project, is provided on opposite sides of the feed element.

The longitudinal guide means is preferably arranged in the clamping jaws. In this configuration it is readily possible that the supply guide means and the clamping jaw guide means are inclined relative to one another, e.g. the angle of inclination between the supply guide means and the clamping jaw guide means can lie between 5° and 45°, preferably between 15° and 30°.

To ensure a displacement of the feed element without jolts, it can be provided that the longitudinal guide means runs in a curve in the transition region between the supply guide means and the clamping jaw guide means.

In this case, it is favourable if the feed element is elastically deformable, so that it can also readily follow a curved longitudinal guide means.

For example, the feed element can be in the form of a strip, the plane of the feed element then preferably lying in the plane of the ligature clip.

In such a configuration it is advantageous if the feed element in the form of a strip bears at least one laterally projecting arm, which projects into a longitudinal groove of the instrument and thus guides the feed element in the longitudinal direction.

Of course, such arms that project into a corresponding longitudinal groove can then also be provided on opposite sides of the band-shaped feed element.

According to a preferred embodiment, at its end the arm bears a guide section bent at right angles, the length of which corresponds to the width of the longitudinal groove. This guide section thus forms a guide shoe that is displaceably guided in the longitudinal groove.

The feed element can simply abut against the rear end of the ligature clip to feed the ligature clips, but it is particularly advantageous if the feed element at least partially encompasses the ligature clip in the abutment region on the ligature clip. As a result, the ligature clip is also secured at the rear end embraced by the feed element against pivoting out of the plane of the ligature clip, and therefore is provided with guidance in this region. The feed element thus assumes a double function, namely the function of feeding and the function of guiding the rear end of the ligature clip. This is advantageous in particular when ligature clips are used, the legs of which diverge slightly starting from the bridge section and thus only abut in the region of the free ends of the clamping jaw guide means, while remaining without guidance in the remaining regions of the leg. In this way, a 3-point guidance is obtained for such ligature clips at the two free ends of the legs and at the abutment region of the feed element, so that the ligature clips are securely guided during the entire feeding movement from the. feed guide means into the clamping jaw guide means.

Ligature clips with diverging legs have the advantage that they provide the surgeon with a larger opening angle for placement on a blood vessel or against any other tissue part. Moreover, in a storage cartridge, in which a number of ligature clips are arranged, these are arranged relatively closely one behind the other and substantially more closely than in the case of ligature clips, in which the legs run parallel to one another and which must therefore adhere to a minimum spacing corresponding to the full structural length of a ligature clip.

To enable this guidance between the feed element and the ligature clip, it can be provided, for example, that the feed element has a feed surface running transversely to the feed direction, a lower support surface abutting against the underside of the ligature clip and an upper support surface abutting against the upper side of the ligature clip.

In particular, in the case of a ligature clip that consists of two sections located next to one another forming a slit between them, the upper support surface can engage into the slit and abut against the upper side of the section, against the underside of which the lower support surface abuts. Thus, in the region of the bridge section the ligature clip is either embraced completely or in the region of a section is embraced on three sides by the feed element and is thus reliably guided.

The following description of preferred embodiments of the invention serves for more detailed explanation in association with the drawing.

### BRIEF DESCRIPTION OF THE DRAWINGS

FIG. 1 is a perspective view of a surgical instrument for the placement of C-shaped ligature clips;

FIG. 2 is a perspective detail view of the transition region from a cartridge into the clamping jaws, wherein for reasons of clarity only one clamping jaw is shown with one ligature clip between the clamping jaws;

FIG. 3 is a view similar to FIG. 2 without a ligature clip between the clamping jaws;

FIG. 4 is a perspective view of the clamping jaw region of FIG. 2 from a different viewing angle;

FIG. 5 is a view similar to FIG. 4 without a ligature clip and with the feed element and clamping jaw illustrated separately; and

FIG. 6 is a sectional view taken along line 6-6 in FIG. 2.

### DETAILED DESCRIPTION OF THE INVENTION

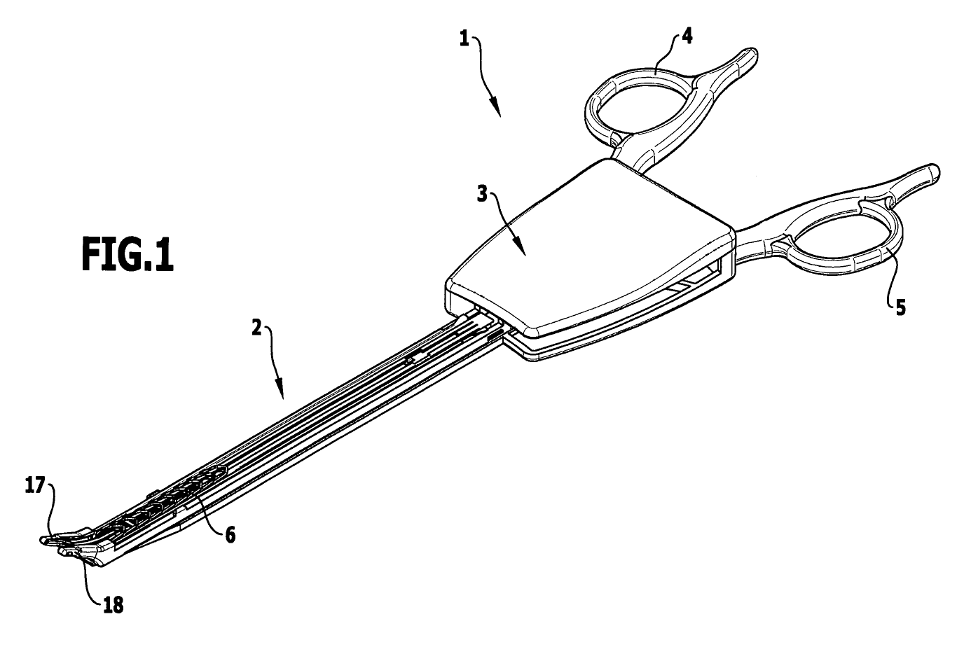

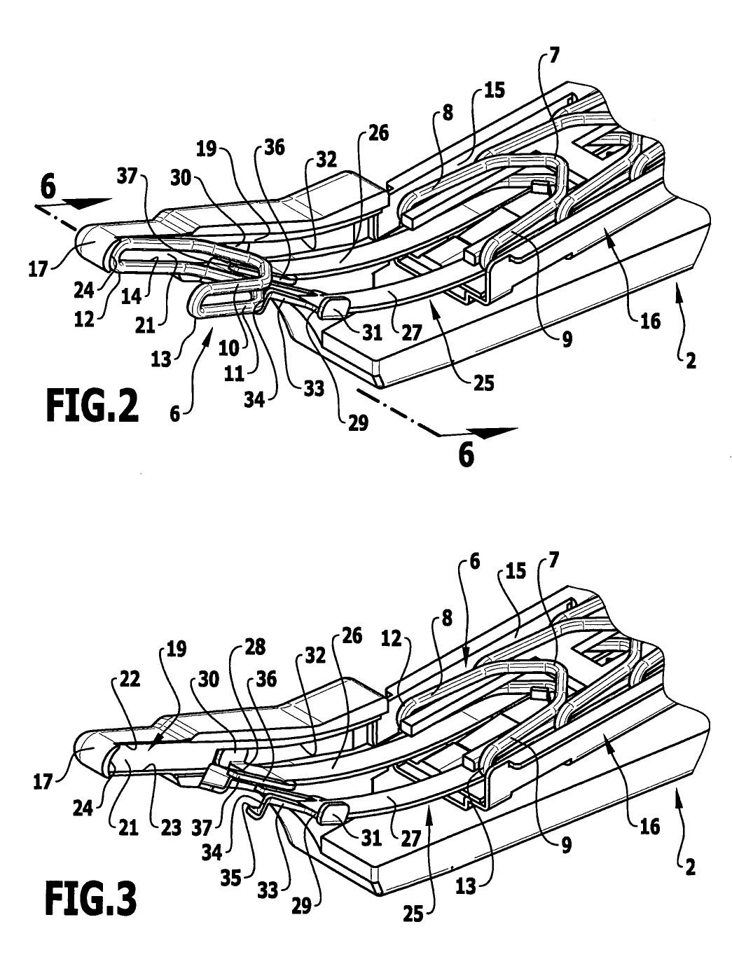

The instrument 1 shown in FIG. 1 comprises an elongated shank 2 with a handle part 3 at its proximal end. Two handle branches 4, 5, which activate different parts of a feeding means in the interior of the shank 2 by means of a gear mechanism, are pivoted to the handle part 3. A cartridge 16, in which a large number of C-shaped ligature clips 6 are arranged one behind the other, is inserted into the shank 2. The ligature clips 6 used in the present exemplary embodiment are C-shaped in configuration and have two legs 8, 9 arranged next to one another, which are connected to one another by means of a bridge section 7 and diverge slightly from the bridge section 7 to the free ends in such a way that the ligature clips 6 can be arranged one behind the other in the cartridge, so that the free ends of the legs 8, 9 pointing in the distal direction are arranged next to the proximal ends of the legs of the ligature clip 6 located in front of them (FIGS. 2 and 3).

The ligature clips 6 consist of two sections 10, 11 located next to one another at a spacing, which are only connected to one another by an arc at the free ends 12, 13 and between them enclose a longitudinal slit 14 extending over the entire length of the legs and the bridge section.

A large number of such ligature clips 6 lie in the shank along a straight guide path 15, which is formed by the side walls of a cartridge 16, for example, against which the outer surfaces of the legs 8, 9 of the ligature clip 6 abut.

This guide path 15 terminates at the distal end of the cartridge 16, which is open at its distal end.

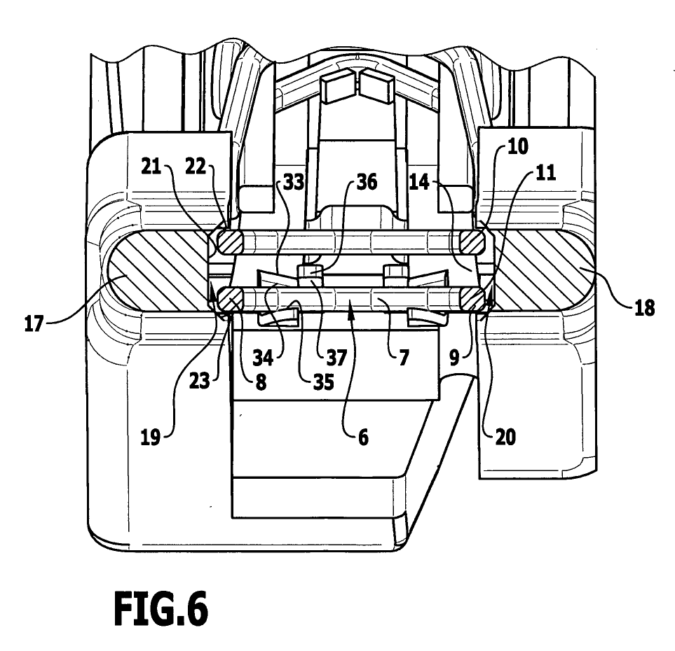

Two clamping jaws 17, 18 located next to one another, which have guide surfaces 19, 20 on their inner surfaces facing one another for ligature clips fed between them, adjoin this distal end of the cartridge 16. These guide surfaces have the shape of a longitudinal groove in cross-section with a base 21 and with lateral guide walls 22, 23, and the ligature clips 6 fed between the clamping jaws project into these guide surfaces 19, 20 with their free ends 12, 13. In this region, the legs 8, 9 abut against the base 21 of the guide surfaces 19, 20 with their outer surfaces. However, because of the diverging arrangement of the legs 8, 9, the contact with the base of the guide surfaces is restricted to the regions directly adjoining the free ends 12, 13, the parts of the legs lying in proximal direction thereto no longer abut against the base 21, but are still guided by the lateral guide walls 22, 23 over a specific length (FIG. 6).

The guide surfaces 19, 20 form a curved guide means, which adjoins the guide path 15 of the cartridge 16 with a curved region and which terminates in a rectilinear end region, which is delimited by an inwardly projecting end stop 24.

A feeding means activated by actuation of the handle branches 4, 5 is provided to advance the ligature clips 6 in the cartridge 16 and out of the cartridge 16 between the clamping jaws 17, 18. This feeding means is divided into two separate elements, one element displaces all the ligature clips 6 in the cartridge 16 on each actuation of the handle branches 4, 5 by one position in distal direction. This feeding means is not shown in the drawing and is likewise not explained in more detail. This is a feeding means that is known per se.

A further feeding means serves to feed the ligature clip 6 furthest forward in each case out of the cartridge 16 along the guide surface 19 between the clamping jaws 17, 18 until the ligature clip 6 abuts against the end stop 24 with its free ends 12, 13 and thus is located in the placement position between the clamping jaws.

This second feeding means comprises a flexible, feed element 25 in the form of a strip, which runs parallel to the plane of the ligature clips 6 in the cartridge 16 and is alternately pushed forward and back upon actuation of the handle branches 4, 5. At its distal end this feed element 25 bears two arms 26, 27 spaced next to one another, which are respectively widened at their free end and there form projections 28, 29, the end sections of which form guide tabs 30, 31 bent at right angles (FIG. 5). These guide tabs 30, 31 project into a respective longitudinal groove 32, which is located in the base 21 of the guide surface 19 of each clamping jaw 17, 18 and follows the course thereof. As a result, the arms 26, 27 are guided at their free ends by the guide tabs 30, 31 engaging into the longitudinal grooves 32 and during the feed movement follow the course of this guide surface 19.

An extension 33 running in the plane of the arms 26, 27 respectively adjoins the free ends of these arms 26 and 27, which extension is bent downwards at its end and thus forms a feed surface 34 running substantially perpendicularly thereto and a lower support surface 35 running parallel to the extension 33. In addition, adjacent to the extension 33 a narrow tongue 36 is separated from the extension 33. This narrow tongue is bent slightly upwards in relation to the extension 33 and its free end forms an upper support surface 37 (FIG. 5).

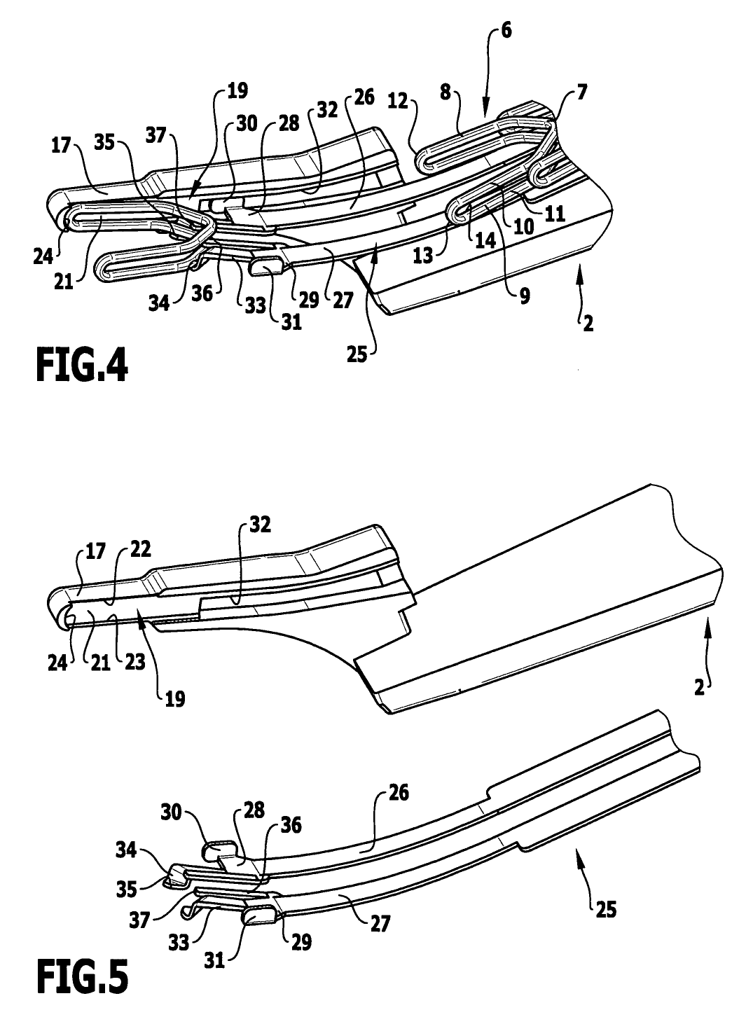

When the feed element 25 advances out of its proximal end position, it abuts against the proximal end of a ligature clip 6 arranged in the front position in the cartridge 16. During this, the feed surfaces 34 abut against the rear side of the bridge section 7, the lower support surface 34 abuts against the underside of the bridge section 7 and the upper support surface 37 engages into the, longitudinal slit 14 of the ligature clip 6 and abuts against the upper side of the respective lower section of the ligature clip 6, so that in the region of the bridge section 7 this lower section of the ligature clip is thus embraced on three sides by the feed element 25 (FIG. 4). As a result, the ligature clip is guided by the feed element 25 in this region, i.e. the bridge section can no longer move out of the plane determined by the arms 26, 27 of the feed element 25, and this plane is in turn determined by the engagement of the guide tabs 30, 31 into the longitudinal groove 32. The ligature clip 6 is thus guided along its entire feed path from the cartridge to the clamping jaws in three regions, namely once in the regions of the free ends 12, 13 of the legs 8, 9, which abut against the guide surface 19 between the two guide walls 22, 23, and in the region of the bridge section, which is embraced by the parts of the feed element 25 in the described manner. Thus, it is possible to securely guide the ligature clips over a large distance and also over a large angle of inclination between the guide path 15 in the cartridge 16 and the guide surface 19 between the clamping jaws 17, 18 over the entire path. Therefore, the instrument can have a clamping jaw region that is steeply inclined in relation to the longitudinal direction of the shank, that is also at a greater distance from the cartridge in the shank and is therefore particularly clearly visible for the surgeon.

The feed element 25 can be made from spring steel and be driven in a reciprocating manner by the handle branches 4, 5 in a manner known per se. When the feed element 25 is pulled back the tongue 36 will actually slide past the ligature clip furthest forward in the cartridge, since the tongue is resiliently pivoted upon abutment against this ligature clip. The same applies with respect to the extension 33, which is likewise resiliently pivoted by the ligature clip when the teed element 25 is pulled back past this ligature clip. During feeding both the extension 33 and the tongue are again located in a position, in which the lower support surface 35 can abut against the underside and the upper support surface 37 can abut against the upper side of the lower section of the ligature clip.

1. A surgical instrument for the placement of C-shaped ligature clips having two legs running next to one another and connected to one another by means of a bridge section, with two clamping jaws movable relative to one another, with a supply guide means, along which the ligature clip can be pushed onto the clamping jaws with the free end of the legs first, with a clamping jaw guide means, which adjoins the supply guide means and in which the ligature clip can be inserted between the clamping jaws, and with a feeding means for displacing a ligature clip out of the supply guide means into the clamping jaw guide means and into an end position between the clamping jaws, wherein the feeding means comprises a feed element, which can be placed against the ligature clip to be fed and is guided in a longitudinal guide means running along its feed path in the transition region from the supply guide means into the clamping jaw guide means.

2. A surgical instrument according to claim 1, wherein the longitudinal guide means is formed by at least one longitudinal groove, into which a lateral guide projection of the feed element projects.

3. A surgical instrument according to claim 2, wherein there is provided, on opposite sides of the feed element, a respective longitudinal groove, into which project lateral guide projections of the feed element located opposite one another.

4. A surgical instrument according to claim 1, wherein the longitudinal guide means is arranged in the clamping jaws.

5. A surgical instrument according to claim 1, wherein the supply guide means and the clamping jaw guide means are inclined relative to one another.

6. A surgical instrument according to claim 4, wherein the supply guide means and the clamping jaw guide means are inclined relative to one another.

7. A surgical instrument according to claim 5, wherein the angle of inclination between the supply guide means and the clamping jaw guide means lies between 5° and 45°.

8. A surgical instrument according to claim 5, wherein the longitudinal guide means runs in a curve in the transition region between the supply guide means and the clamping jaw guide means.

9. A surgical instrument according to claim 1, wherein the feed element is elastically deformable.

10. A surgical instrument according to claim 1, wherein the feed element is in the form of a strip.

11. A surgical instrument according to claim 10, wherein the feed element in the form of a strip bears at least one laterally projecting arm, which projects into a longitudinal groove of the instrument and thus guides the feed element in the longitudinal direction.

12. A surgical instrument according to claim 11, wherein at its end the arm bears a guide section bent at right angles, the length of which corresponds to the width of the longitudinal groove.

13. A surgical instrument according to claim 1, wherein the feed element at least partially embraces the ligature clip in the abutment region on the ligature clip.

14. A surgical instrument according to claim 13, wherein the feed element has a feed surface running transversely to the feed direction, a lower support surface abutting against the underside of the ligature clip and an upper support surface abutting against the upper side of the ligature clip.

15. A surgical instrument according to claim 14, wherein in the case of a ligature clip consisting of two sections located next to one another forming a slit between them, the upper support surface engages into the slit and abuts against the upper side of the section, against the underside of which the lower support surface abuts.

16. A surgical instrument according to claim 1, wherein the legs of the ligature clip diverge from the bridge section to the free ends of the legs, so that the legs are only guided at their fee ends in the clamping jaw guide means.
